(19) United States
(12) Patent Application Publication
(10) Pub. No.: US 2017/0348208 A1
(43) Pub. Date: Dec. 7, 2017

(54) CARIES-RESISTANT COMPOSITE RESIN

(71) Applicant: Den-Mat Holdings, LLC, Lompoc, CA (US)

(72) Inventors: Jeff MacDonald, Lompoc, CA (US); Alvin Kobashigawa, Mission Viejo, CA (US)

(21) Appl. No.: 15/613,164

(22) Filed: Jun. 3, 2017

Related U.S. Application Data
(60) Provisional application No. 62/345,578, filed on Jun. 3, 2016.
(60) Provisional application No. 62/434,175, filed on Dec. 14, 2016.

(51) Int. Cl. A61K 8/27; A61K 8/41; A61K 8/35; A61Q 11/00; A61K 8/22

(52) U.S. Cl. CPC A61K 8/27; A61Q 11/00; A61K 8/22; A61K 8/355; A61K 8/411

(57) ABSTRACT

A single and dual component dental composite restorative featuring anti-microbial and low Coefficient of Thermal Expansion (CTE) compounds is disclosed. The exemplary anti-microbial compound is zinc oxide. The CTE of the dental composite restorative is the same as, or substantially similar, to that of dentin. By maintaining a CTE substantially similar to that of dentin, the Margin Percolation phenomenon is minimized, which decreases the incidence of secondary caries.

### CROSS-REFERENCE TO RELATED APPLICATIONS

This application claims the benefit of U.S. provisional patent applications Ser. No. 62/345,578, filed Jun. 3, 2016, entitled “Caries-Resistant Composite Resin” and Ser. No. 62/434,175. Entitled “Caries-Resistant Composite Resin”, the contents of which are incorporated herein by reference.

### FIELD OF THE INVENTION

The present invention relates to improvements in tooth treatment compositions. In particular, this invention relates to dental composite restoratives with increased resistance to secondary caries formation.

### BACKGROUND OF THE INVENTION

Composite resins were first introduced to dentistry in the late fifties. They contained particulate fillers and had improved physical properties over unfilled resins. The polymerization shrinkage was lower and long-term clinical performance looked promising, especially with respect to wear resistance and reduced secondary caries. In the early sixties, bis-GMA as a resin matrix, which further improved the properties of composite resins. However, high wear rates were observed clinically in posterior restorations. Because of this, composite resin research has been devoted to improving wear resistance. In time, the wear resistance improved and in the early eighties, and the use of composite resins in posterior restorations became acceptable. Today, the wear resistance of composite resins typically exceeds ten (10) years and composite resins are now routinely used in posterior restorations.

Despite the improvements in wear resistance in composite resins, secondary caries continues to be a problem for composite resins throughout its existence. As an illustration, modern composite resins still have an average clinical life of about 8-10 years. However, the real-world service life goes down to about 5.7 years. The shorter service life is attributed to the development of secondary caries along with bulk failures. Secondary caries are caries that occur on a tooth after a restorative has been used. When a secondary caries occurs, restoratives must be replaced.

In order to reduce secondary caries in composite resins, several research strategies were employed in the past. One strategy was to reduce the polymerization shrinkage. Shrinkage stress between the tooth and restoration was thought to be a major reason for gap formation, resulting in caries formation within the gaps. To reduce shrinkage, composite resins with particulate fillers were developed, which lowered the shrinkage by about 50% (by reducing the total resin content). This was a main reason for optimism in improving clinical performance. Shrinkage has also been reduced in the resin matrix itself, for example with monomers that expand on polymerization. Together, volume polymerization has been reduced from about 3% to less than 1% in composite resins. However, the improvement in shrinkage has not lead to a corresponding reduction in reduced secondary caries.

Another strategy was to develop adhesives to maintain the bond integrity between the tooth and restoration. It was believed that if a strong bond between the tooth and restoration could be established, gap formation would be reduced and secondary caries formation would also be reduced. In the mid 1950s, adhesion to tooth enamel was accomplished by using a technique where phosphoric acid etched the enamel. Using this technique resulted in a bond strength to enamel of about 20 MPa and was clinically stable. However, this technique alone did not prove to be effective in reducing secondary caries despite this bond strength. In addition to the bond strength with the enamel, adhesion to dentin and cementum was also thought to be necessary.

Improving adhesion to dentin and cementum has been a major research focus since. At least eight generations of adhesives have been developed. The first generation started in the mid 1950s where the composite resin contained a dentin coupling agent, glycerophosphoric acid dimethacrylate. The bond strength to dentin proved to be very erratic because of the hydrophobic resin matrix. Today, in the eighth generation of composite resins, higher more consistent bond strengths have been achievable, in the 20-30 MPa range. However, despite being able to more consistently achieve high bond strengths, over time the bond strength deteriorates. As a result, the improved bond strengths have not lead to a corresponding improvement of the secondary caries rate.

Yet another approach to reducing secondary caries in composite resins has been to incorporate antimicrobials. This approach was logical since there is a history of caries-resistant materials containing antimicrobials in dentistry. For example, zinc phosphate and zinc oxide eugenol are compounds that exhibit anti-microbial behavior and have been incorporated into restoratives and cements. Silver, which is a component in amalgam, also has antimicrobial activity. However, the long-term caries resistance effect of amalgams has not been proven to be effective.

Other antimicrobial compounds such as quaternary ammonium compounds and antibiotics have been incorporated into adhesives and composite resins. Examples of quaternary ammonium compounds include Cetylpyridinium chloride (CPC), Methacyloyloxdodecylpyridinium (MDPB), Poly-quaternary ammonium salts (PQAS) and methacryloxylethyl cetyl dimethyl ammonium chloride (DMAE-CB). Antibiotics such as ciprofloxacin, minocycline, cefaclor and metronidazone have been added directly to composite resins. While these materials do inhibit bacterial growth for various periods, but still there are no clinical studies that show a significant reduction in secondary caries.

Despite the research, secondary caries in composite resin restorations continues to be a problem today. This is the main reason why the clinical life of these restorations has not improved. Restorative materials such as zinc phosphate and zinc oxide eugenol have proven to be effective in reducing secondary caries, but other restorative materials, including composite resins and amalgams, have poor records. So, there exists a need for a direct fill dental composite resin that reduces the incidence of secondary caries.

### BRIEF DESCRIPTION OF THE INVENTION

The present invention deals with a novel dental restorative composition that features resistance to the formation of secondary caries. This dental restorative composition can be either a single component formulation, or a dual component formulation that is admixed prior to use.

The secondary caries resistance of this novel dental restorative composition is a result of the incorporation of an anti-microbial compound, and the use of compounds that exhibit a low Coefficient of Thermal Expansion (CTE).

The inclusion of an antimicrobial compound in the dental restorative composition will decrease the incidence of bacterial growth on the dental restorative. By reducing the incidence of bacterial growth, secondary caries are reduced.

The use of compounds that exhibit a low CTE reduce the occurrence of the phenomenon known as “Marginal Percolation.” Marginal Percolation occurs when the dental restorative and dentin expand or contract at different rates, due to a difference of CTE. If the CTE difference is substantial enough, the difference in expansion and contraction will form a cap between the dentin and restorative. Bacteria, can then enter the resulting gap and cause secondary caries.

Thus, the inclusion of both an antimicrobial and low CTE compounds, taken together, results in a reduction of secondary caries in dental restorations.

### DETAILED DESCRIPTION OF THE INVENTION

Some dental materials have performed well in terms of inhibiting secondary caries when used as a restorative. Gold restorations for example, have performed best, often lasting a lifetime clinically. Gold foil restorations adapt well and perform well without adhesives. Gold crowns also perform well with proven refractory cements. Probably the most important feature of gold as a dental material is its Coefficient of Thermal Expansion (CTE).

Ideally, the CTE of dental materials should match the CTE of dentin, or be substantially similar to the CTE of dentin. When this happens, the restorative and tooth expand and contract at the same rate as the temperature of the oral cavity cycles. This prevents stress from developing at the interface at the bond between the restorative and dentin, and helps maintain an integral bond between the materials. More importantly, a matched CTE between the materials will prevent a phenomenon referred to as “Marginal Percolation”. “Marginal Percolation” is a phenomenon whereby a gap is formed in between the tooth and restorative. This gap is formed because the dentin and restorative are expanding or contracting at different rates, allowing a continuous influx of bacteria and nutrients for established bacterial colonies into the gap. As discussed below, there is a large difference in CTE between the typical dental restoratives and dentin (for example composites and amalgams have a CTE of about 30 ppm/deg. C. vs. 12 ppm/deg. C. for dentin). With temperature cycling, “Marginal Percolation” occurs and bacteria and nutrients percolate into the gap allowing bacterial growth. The existence of growing bacteria within the formed gap results in secondary caries. On the other hand, if the dentin and restorative are expanding and contracting at the same rate, this gap is not exposed, resulting in less bacterial growth, and a corresponding decrease in secondary caries are formed less readily.

Operator error often results in marginal gaps between the tooth and restoration. Clinical studies have shown that more than 50% of restorative failures are a result of operator error. This is a matter of operator technique and happens no matter how good the restorative or adhesive is. In the situation where gaps occur and where there is a large difference in CTE between the tooth and restoration, “Marginal Percolation” occurs within the gaps. Case 1 in FIG. 1 illustrates this when there is a large difference in CTE between the restoration and tooth (for example composites and amalgams have a CTE of about 30 ppm/deg. C. vs. 12 ppm/deg. C. for dentin). With temperature cycling, “Marginal Percolation” occurs and bacteria and nutrients percolate into the gap allowing bacterial growth.

Table 1 shows the CTE of some relevant materials in accordance to the international standard ASTM Method D696. The CTE of dentin is approximately in the range of 14-17. So, the materials with the closest match would ideally be within that same range of 10-15, most preferably as close to the CTE of dentin as possible. Note that the materials with matched or lower CTEs to the tooth have a history of low caries incidence, whereas amalgams and composites have high CTEs and a history of high secondary caries incidence. Note also that these materials do not use adhesives and have little or no bonding to the tooth. This suggests that except for retention, the adhesive bond may not be as important a factor in preventing secondary caries as previously believed.

TABLE 1
The coefficient of thermal expansion (CTE) of various
restorative materials.

| | COEFFICIENT OF THERMAL |
|---|---|
| RESTORATIVE MATERIAL | EXPANSION (CTE) ppm/° C. |
| MATCH | |
| Gold | 14 |
| PFM | 14 |
| Porcelain | 14 |
| Silicate cement | 7.6 |
| Glass ionomer cement | 9 |
| Zinc phosphate cement | 4.6 |
| POOR MATCH | |
| Amalgam | 30 |
| Composite resin | 30 |
| Zinc Oxide Eugenol | 35 |

The dental restorative disclosed below decreases the incidence of secondary caries by taking advantage of two novel features that must occur together. First, this novel dental restorative includes a compound with antimicrobial properties that are long lasting clinically. The activity does not have to be strong, but must be long lasting. For example, zinc oxide has mild antimicrobial activity, but appears to be long lasting; whereas silver has strong activity, but may not last as long. However, antimicrobial activity alone does not necessarily guarantee caries resistance. Amalgams, for example, contain silver and demonstrate good antimicrobial activity initially, but in the long term have proven to have a high rate of caries incidence. To be effective, caries-resistant materials must have antimicrobial activity throughout its restorative life.

Enduring antimicrobial activity may be related to the second important feature, ensuring that the dental restorative has a CTE that matches or is at least substantially similar to the CTE of dentin. As discussed, when this happens, the “Marginal Percolation” effect does not happen and bacterial penetration and dilution of antimicrobials is minimized. The “Marginal Percolation” effect may help explain why some materials such as amalgams, lose their caries resistance over time. As illustrated in Table 1, silver in amalgams has strong antimicrobial activity, but amalgams also have a higher CTE value than the tooth. As a consequence, “Marginal Percolation” promotes dilution of the antimicrobial and also promotes bacterial penetration into the marginal gaps.

Unfortunately, the importance of a matched CTE to the tooth has been marginalized in the past. The reason for this is that the difference is relatively small. All direct filling composites have a CTE of about 30 ppm/° C. vs. about 12 ppm/° C. for dentin (the high CTE is the result of the high CTE of polymers, about 80 ppm/° C.). The difference in CTE is small and probably has little effect on development of bond stresses compared to stress developed by polymerization shrinkage, which had been the focus of dental restorative research since the 1950s. As a result, the focus of past research has been to reduce polymerization shrinkage instead of matching the CTE.

Also reducing the CTE of composite resins has not been achieved due to the reliance on already existing materials used in dental restoratives. Most polymers used in dental restoratives have a CTE in the range of about 80 ppm/° C. Polymers with aromatic backbone such as BisGma have lower CTE of about 60 ppm/° C. Addition of inorganic fillers has reduced the CTE of composite resins to about 30 ppm/° C., However, further reductions in CTE to match the tooth has not been explored in the current landscape of dental restorative materials. As a result, from a materials standpoint, there has been an absence of materials to formulate dental composite resins which are effective at being a restorative material, and have a CTE which is within the range of dentin.

As a result, there is a long-felt need for a direct composite resin filling material that include both antimicrobial properties and a CTE that matches closely to the tooth itself to decrease the incidence of secondary caries. Zinc oxide is a compound that exhibits antimicrobial properties and lowers the CTE of a dental restorative to match dentin at high filler loading.

As an antimicrobial compound, zinc oxide has several advantages. Through testing by international standard AATTCC 100 TM-2012, zinc oxide has a long history of mild antimicrobial activity and long-term effectiveness (see Table 2). However, zinc oxide is not typically thought of as a chemical compound for antimicrobial use in the oral cavity. The most well-known commercial use of zinc oxide is for its UV absorption properties in sunscreen.

TABLE 2
Effect of Zinc Oxide on the antimicrobial activity of a direct
filling composite resin. Microbial activity is measured using
the AATCC TM 100-2012 standard.

| Wt. Fract. ZnO | % Reduction of e coli | % reduction of strep mutans |
|---|---|---|
| 0 | 0% | 0% |
| 1.0% | 100% | 100% |

When zinc oxide is incorporated into a chemical for use in the oral cavity, it is typically incorporated for its function as an opacifying agent and can be added to a product for this function alone. However, zinc oxide is not considered an ideal opacifying agent for dental restorative compositions because the color of zinc oxide is a chalky-white color, which is not the ideal color for dental restoratives. As such, there is a limit to the amount that can be added. Resins are esthetic restorative materials and require high translucency to be esthetic. For this reason, the amount of Zinc Oxide that may be added is limited to less than 0.8 wt. fract. (Table 3).

TABLE 3
Effect of Zinc Oxide on the translucency of a direct filling
composite resin.

| Wt. Fract. ZnO | Translucency |
|---|---|
| 0 | pass |
| 0.8 | pass |
| 1.0 | fail |

In Table 3, as the wt. fraction of Zinc Oxide is increased, the opacity also increases to a level that is not considered suitable for use as a dental composite resin for esthetic purposes. Beyond this level, the opacity becomes too high and the restoration appears a chalky white. Additionally, there exists commonly used compounds that exhibit superior opacifying properties exists, such as titanium dioxide. Thus, zinc oxide is not a compound that is typically found in dental compositions.

Dental restoratives used in core build up procedures are typically lower filled composite restoratives, used in internal portions of the teeth, where esthetics (color, translucency, surface finish) and wear properties are less important, and mechanical strength (>115 megapascals in flexural strength), microbial and CTE issues are of primary importance. Because of the decreased emphasis on esthetics for these types of dental restoratives, higher amounts, for example greater than 0.8%, of zinc oxide can be used. These materials are used to fill irregular, often small, undercut internal voids in the dentition, as well as being used to support implantable synthetic “core posts” (metal or fiber rods of 0.5-2 mm in diameter and 1-5 mm in length). Due to these variable uses, the viscosity of this class of materials is lower, not too dissimilar to that of an over the counter dentifrice.

This class of materials are often light cured, chemically/“self” cured (via benzoyl peroxide curatives) as well as dual cured, allowing for light and self-cure with the same material. The material discussed here is dual cure, allowing for a fast cure via photo initiation, or allowing the material to cure more slowly on its own. When the composition is light cured, a photo initiator compound is included into the composition. A fairly typical photo initiator used in light cured dental composite restoratives is camphorquinine. However, any other suitable photo initiator can be used.

In order to prevent premature self-curing, this material is typically a two-component composition that are stored separately to allow the materials to maintain the desired viscosity (and not cure) and are admixed at the time of the dental procedure. The need for mixing as well as the aforementioned broad scope of use as a core material requires that the viscosity be relatively low, when compared to other conventional composite restoratives. Each component will contain compounds that are chemically inert by itself, however, when combined, will cure. In the embodiment discussed below in Table 4, component A contains benzoyl peroxide and component B contains dihydroxyethyl paratoluidine. When these chemicals are combined, the resulting chemical reaction will cure the resulting composition. Any other suitable binary curing system can be used instead of the combination of benzoyl peroxide and dihydroxyethyl paratoluidine.

In particular, this dental restorative is a material that is stored in a dual chamber syringe, and is dispensed through a static mixer tip, and is intended to be dispensed by hand pressure alone (no mechanical aids such as a cartridge dispensing gun, or other devices that provide mechanical advantage and reduce the force needed to dispense) As such, there is an implicit maximum to how thick the material can be, and still be practicably dispensed from the packaging by dental professionals, including those with below average hand strength.

Additionally, this dental restorative can also encompass the novel inclusion of bioactive components into the formulation. Bioactivity is determined in accordance with ISO/FDIS 23317. Such bioactive components allow for the formation of hydroxyapatite (the primary component of natural teeth) and allows the restoration to improve long term retention via the remineralized restoration surface “growing” into a single cohesive part of the tooth. As those skilled within the art are aware, the addition of bioactive materials imparts reduced mechanical properties of the final cured material.

The need for strong mechanical properties, as well as lower viscosity requirements, the bioactivity requirements, low CTE and antimicrobial components, provide a difficult set of desired properties that each can reduce the other properties.

An exemplary two-component dental restorative that incorporates zinc oxide and bioactive materials to yield a novel composite resin that exhibits antimicrobial materials and a CTE that is in the desired range to mimic dentin is as follows:

TABLE 4
Example 1
Part A

| | Weight/weight |
|---|---|
| Chemical | percentage |
| Benzoyl peroxide | 0.15-0.30 |
| diphenyl (2,4,6-trimehylbenzoyl) phosphine oxide | 0.07-0.09 |
| Ethyl 4-dimethylaminobenzoate | 0.05-.15 |
| Camphorquinone | 0.05-0.15 |
| ethoxylated bisphenol A dimethacrylate | 12-18 |
| Bis GMA | 2-5 |
| triethylene glycol dimethacrylate | 8-12 |
| 2-(2′hydroxy-5′-octylphenyl) benzotriazole | 0.4-0.5 |
| butylated hydroxytoluene | 0.008-0.01 |
| barium glass | 33-47 |
| low CTE glass | 8-12 |
| fumed silica-hydrophobic | 0.5-1 |
| fumed silica-hydrophyllic | 0.1-0.5 |
| zinc oxide | 0.2-0.75 |
| Calcium fluoride | 0-1.75 |

Part B

| | Weight/weight |
|---|---|
| Chemical | percentage |
| dihydroxyethyl paratoluidine | 0.6-0.9 |
| diphenyl (2,4,6-trimehylbenzoyl) phosphine oxide | 0.07-0.09 |
| Ethyl 4-dimethylaminobenzoate | 0.05-.15 |
| Camphorquinone | 0.05-0.15 |
| ethoxylated bisphenol A dimethacrylate | 12-18 |
| Bis GMA | 2-5 |
| triethylene glycol dimethacrylate | 8-12 |
| 2-(2′hydroxy-5′-octylphenyl) benzotriazole | 0.4-0.5 |
| butylated hydroxytoluene | 0.008-0.01 |
| barium glass | 33-47 |
| low CTE glass | 8-12 |
| fumed silica-hydrophobic | 0.5-1 |
| fumed silica-hydrophyllic | 0.1-0.5 |
| zinc oxide | 0.2-0.75 |
| Calcium fluoride | 0-1.75 |
| Hydroxyapatite | 3-7 |
| Bioactive glass | 9-12 |

In an alternative embodiment would be a single component dental restorative that is light cured only. Because this embodiment lacks the ability to self-cure the entire composition can be stored in a single component. However, lack of self-curing reduces the dental professional's flexibility in use.

TABLE 5
Example 2

| | Weight/weight |
|---|---|
| Chemical | percentage |
| diphenyl (2,4,6-trimehylbenzoyl) phosphine oxide | 0.07-0.09 |
| Ethyl 4-dimethylaminobenzoate | 0.05-0.15 |
| Camphorquinone | 0.05-0.15 |
| ethoxylated bisphenol A dimethacrylate | 12-18 |
| Bis GMA | 2-5 |
| triethylene glycol dimethacrylate | 8-12 |
| 2-(2′hydroxy-5′-octylphenyl) benzotriazole | 0.4-0.5 |
| butylated hydroxytoluene | 0.008-0.01 |
| barium glass | 33-47 |
| low CTE glass | 8-12 |
| Bioactive glass | 9-12 |
| fumed silica-hydrophobic | 0.5-1 |
| fumed silica-hydrophyllic | 0.1-0.5 |
| hydroxyapatite | 3-7 |
| zinc oxide | 0.2-0.75 |
| Calcium fluoride | 0-1.75 |

The method of manufacturing such a restorative is as follows

1. The filler glasses and fumed silica are pre-blended

2. The pre-blended filler glasses and fumed silica are added to the resin in small increments until the desired viscosity of the dental restorative is achieved.

3. The ensuing composition is then placed in a roll mill.

Low CTE was achieved by employing a low CTE x-ray radiopaque filler at high loading. For example, using a filler with a CTE value of <1 ppm/° C. vs. a more typical x-ray radiopaque filler which as a CTE of about 4 ppm/° C. The lower CTE value allows further reduction of the CTE of the composite resin to about 15 ppm/° C., well within the range of dentin.

Zinc oxide is within the resin to provide enhanced antimicrobial activity. The amount of zinc oxide incorporated within the resin is sufficiently high enough to provide enhanced antimicrobial activity, but low enough to prevent opacity of the restorative.

Table 6 is a comparison of the CTE between Nuance® (high viscosity Universal Composite restorative) by Den-Mat Holdings, LLC and Examples 1 and 2 as described by Tables 4 and 5 respectively. Nuance was selected for the comparison because it's properties are representative of the current market of dental restoratives. The CTE was measured using the ASTM D 695 standard.

TABLE 6
Coefficient of thermal expansion (CTE) of direct filling
composite resins

| MATERIAL | CTE ppm/° C. |
|---|---|
| Nuance ® | 36.9 |
| Examples 1 and 2 | 16.1 |

Because, the caries-resistant direct filling composite must also function as a restorative material, its material characteristics must meet or exceed the current standards set by currently available dental restoratives. Table 7 compares the flexural strength between the Examples 1 and 2 presented in Tables 4 and 5 respectively, and Nuance®. The flexural strength was measured in accordance with the ISO 4049. As Table 7 indicates, the flexural strength of Examples 1 and 2 exceeds that of Nuance®.

TABLE 7
Flexural strength of composite resins

| MATERIAL | Flexural Strength, Mpa |
|---|---|
| Nuance ® | 83.4 MPa |
| Examples 1 and 2 | >113.0 MPa |

The foregoing description of the invention with the accompanying examples is not intended to be limiting. It is contemplated that other embodiments may be made without departing from the spirit or scope of the invention as set forth in the appended claims.

What is claimed is:

1. A dental composite restorative composition comprising of:
an antimicrobial agent;
a bioactive glass;
hydroxyapatite;
a photo initiator; and
a low coefficient of thermal expansion glass;
wherein the coefficient of thermal expansion of said dental restorative composition is substantially similar to the coefficient of thermal expansion of dentin.

2. The dental composite restorative composition of claim 1 wherein said antimicrobial agent is comprised of zinc oxide.

3. The dental composite restorative composition of claim 2 wherein said zinc oxide is in the range of 0.2-1.5 w/w %.

4. The dental composite restorative composition of claim 1 wherein said coefficient of thermal expansion is in the range of 10-15 ppm/deg. C.

5. The dental composite restorative composition of claim 1 wherein said coefficient of thermal expansion is in the range of 14-17 ppm/deg. C.

6. The dental composite restorative composition of claim 1 further comprising a flexural strength greater than 83.4 MPa.

7. The dental composite restorative composition of claim 1 wherein said light activated curing agent is comprised of camphorquinone.

8. The dental composite restorative composition of claim 1 further comprising radiopaque filler material.

9. The dental composite restorative composition of claim 8 where said radiopaque filler material further comprises barium glass.

10. A dual component dental composite restorative composition comprising:
a first component comprising of a first component of a binary curing system, a low coefficient of thermal expansion glass, and an anti-microbial agent;
a second component comprising of a second component of a binary curing system, a low coefficient of thermal expansion glass, zinc oxide, hydroxyapatite and bioactive glass;
wherein both components are admixed prior to use to form said dental composite restorative composition with a coefficient of thermal expansion substantially similar to the coefficient of thermal expansion of dentin.

11. The dental restorative composition of claim 10 further comprising a flexural strength greater than 83.4 MPa.

12. The dental restorative composition of claim 10 further comprising a light curing agent.

13. The dental composite restorative composition of claim 10 wherein said coefficient of thermal expansion is in the range of 10-15 ppm/deg. C.

14. The dental composite restorative composition of claim 10 wherein said coefficient of thermal expansion is in the range of 14-17 ppm/deg. C.

15. The dental composite restorative composition of claim 10 wherein said antimicrobial agent is comprised of zinc oxide.

16. The dental composite restorative composition of claim 15 wherein said zinc oxide is in the range of 0.2-1.5w/w %.

17. The dental composite restorative composition of claim 1 wherein said first component of said binary curing system is comprised of benzoyl peroxide.

18. The dental composite restorative composition of claim 1 where said component of said binary curing system is comprised of dihydroxyethyl paratoluidine.
